(19) United States
(12) Patent Application Publication
(10) Pub. No.: US 2006/0135785 A1
(43) Pub. Date: Jun. 22, 2006

(54) Alpha-phenyl acetanilide derivatives having an acat inhibiting activity and the therapeutic application thereof

(75) Inventors: Jean-Francois Patoiseau, Castres (FR); Jean-Marie Autin, Labruguiere (FR); Andre Delhon, Castres (FR); Didier Junquero, Burlats (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Pierre Fabre Medicament, Boulogne-Billancourt (FR)

(21) Appl. No.: 10/531,234

(22) PCT Filed: Oct. 15, 2003
(86) PCT No.: PCT/FR03/03038

§ 371 Date: Oct. 12, 2005

(30) Foreign Application Priority Data
Oct. 16, 2002 (FR) 0212855

(51) Int. Cl. C07D 257/02

(52) U.S. Cl. 548/253; 564/162

(57) ABSTRACT

The present invention relates to novel analide derivatives of formula I, enantiomers and stereoisomers thereof, and their pharmaceutically acceptable salts. The invention also relates to pharmaceutical compositions containing the compounds and methods of treating hypercholesterolemia and atherosclerosis therewith.

### CROSS-REFERENCE TO RELATED APPLICATIONS

This application claims the benefit of the filing date of PCT application PCT/FR2003/003038 filed Oct. 15, 2003, which claims priority from French patent application 02/12855 filed Oct. 16, 2002, the disclosures of which are hereby incorporated by reference.

### BACKGROUND OF THE INVENTION

ACAT-inhibiting compounds have previously been identified by the applicant (Patent WO 97/19918). They have blood cholesterol-lowering and antioxidant properties that make it possible to act both on the quantity and the quality of lipids, thus reducing their atherogenic potential and their long-term harmful effects on the vascular wall. However, these compounds have a low bioavailability and a sensitivity to oxidation that limits the use of formulating agents liable to improve their bioavailability.

Compounds having a heterocyclic structure of a tetrazole nature have been described for their ACAT-inhibiting properties and their blood cholesterol-lowering effect (WO 93/04052).

### SUMMARY OF THE INVENTION

The subject of the present invention is directed toward obtaining novel derivatives having an activity profile comparable to those described by the applicant (WO 97/19918), with increased bioavailability and increased chemical and metabolic stability.

### DETAILED DESCRIPTION

The compounds of the present invention correspond to general formula I:

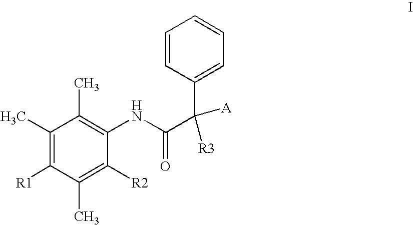

in which:

R₁ represents a hydroxyl or amino group,

R₂ represents hydrogen or a methyl radical,

R₃ represents hydrogen or a fluorine atom,

A represents a group

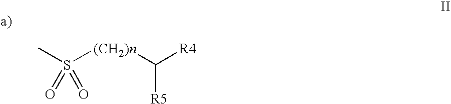

in which:

n represents an integer from 5 to 11, limits inclusive,

R₄ and R₅, which may be identical or different, represent, independently of one another, hydrogen or a fluorine atom

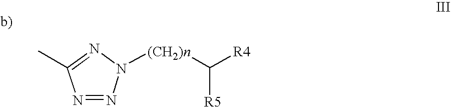

in which n, R₄ and R₅ have the same meaning as above.

Since the compounds of general formula I have one or more asymmetric centers, the present invention covers the various stereoisomers or enantiomers, and mixtures thereof. These can be obtained by conventional methods such as, for example, chromatographic separation on a chiral column.

The present invention also covers the therapeutically acceptable inorganic or organic salts of the compounds of general formula I that have a salifiable function (R₁=amino). The compounds of general formula I can be used for preparing pharmaceutical compositions or medicinal products intended for the treatment of diseases such as hypercholesterolemia and atherosclerosis.

The compounds of the present invention exhibit, unexpectedly, a blood cholesterol-lowering activity in vivo that is greater than the compounds previously described.

Synthesis of the Compounds of Formula I:

The compounds of general formula I can be obtained by treatment of an aniline IV, optionally in hydrochloride form, with the derivative V, the groups R₁, R₂, R₃ and A having the same meaning as above, in the presence of an activator such as dicyclohexylcarbodiimide or 2-chloro-1-methylpyridinium iodide and of triethylamine.

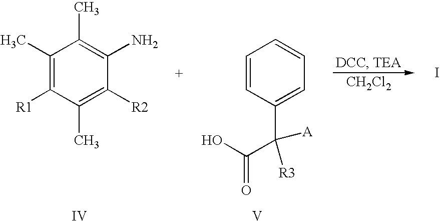

The aromatic amines IV are commercial or can be obtained by methods of synthesis known to those skilled in the art.

The compounds I for which A represents the group II as defined above, with R₁=OH and R₃=hydrogen, can be obtained from the corresponding thioether VI (prepared according to patent WO 07/19918) by oxidation with oxone in aqueous acetone. 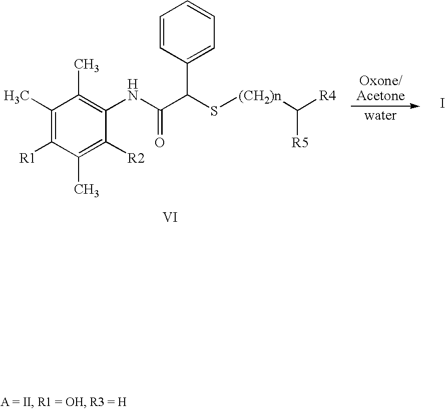
Synthesis of the Compounds of Formula V:

The compounds of formula V for which A represents the group II as defined above and R₃=hydrogen can be obtained by oxidation of the ester VII with a peracid such as m-chloroperbenzoic acid in dichloromethane, followed by alkaline hydrolysis.

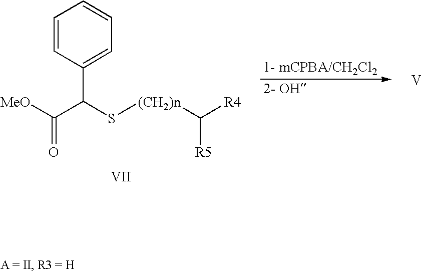

The compounds VII for which R₄ and R₅ represent a fluorine atom can be prepared by DAST fluorination of the bromoaldehyde VIII and then reaction of the derivative obtained on the thiomandelic ester IX.

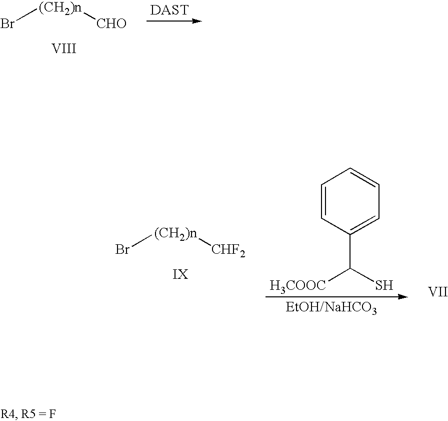

The compounds of general formula V for which A represents the group II as defined above and R₃ represents a fluorine atom can be obtained from the ester of the derivative V in which A=II and R₃=H by treatment with sodium hydride in THF and then with select-fluor (1-chloromethyl-4-fluoro-1,4-diazabicyclo[2.2.2]octane bis(tetrafluoroborate)] in DMF, followed by alkaline hydrolysis.

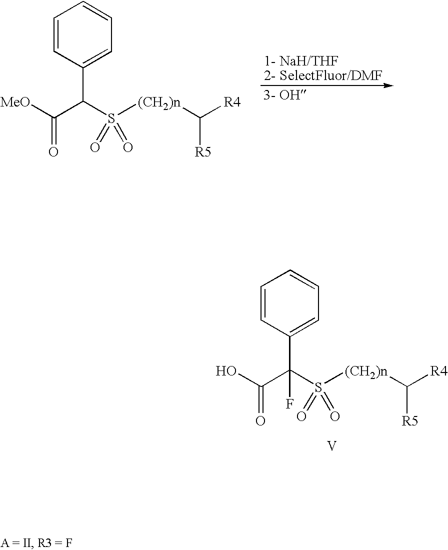

The compounds of formula V for which A represents the group III as defined above and R₃=hydrogen can be obtained according to known methods, for example J. Med. Chem. 1996, 39, 2354-2366.

The compounds of formula V for which A represents the group III as defined above and R₃=fluorine can be obtained from the derivative X and treatment with a base such as sodium hydride in THF and then select-fluor in DMF, followed by alkaline hydrolysis.

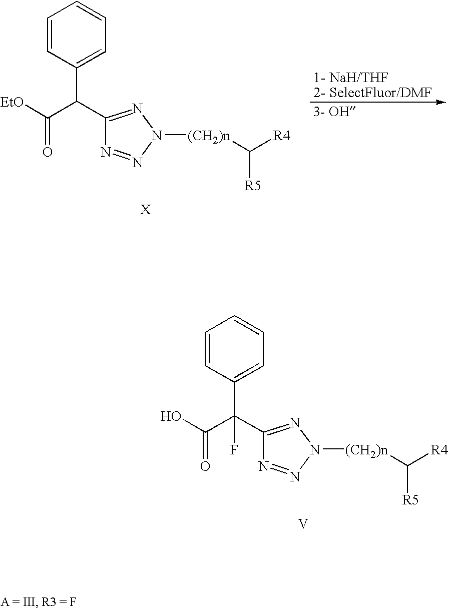

The compounds of formula V for which A represents the group III as defined above, and R₄ and R₅ are fluorine atoms, can be obtained by treating the ester XI with the brominated derivative IX in acetonitrile in the presence of triethylamine, followed by alkaline hydrolysis.

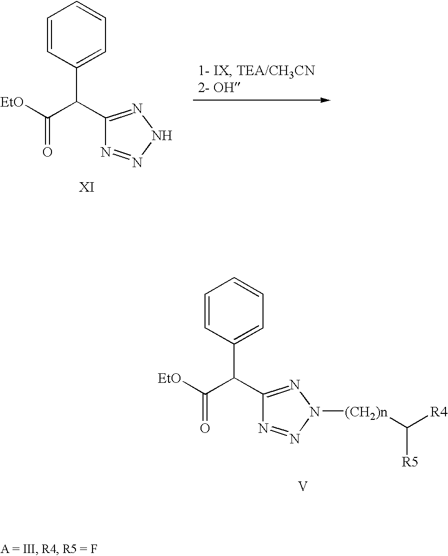

The invention may be illustrated by means of the nonlimiting examples which follow and which constitute advantageous embodiments of the compounds of the invention.

### EXAMPLES

### Example 1

### (S)-2′,3′,5′-Trimethyl-4′-hydroxy-α-dodecylsulfonyl-α-phenylacetanilide 1

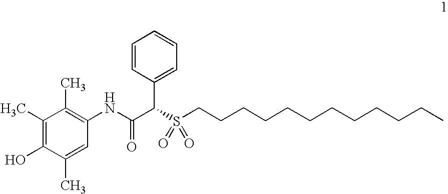

A solution of oxone (32.43 g; 0.053 mol) in water (150 ml) is added, in one go, to a solution of 2′,3′,5′-trimethyl-4′-hydroxy-α-dodecylthio-α-phenylacetanilide (23.5 g; 0.05 mol) in acetone.

After 24 hours at ambient temperature with stirring, the solution is filtered, evaporated to dryness then taken up with ethyl acetate (800 ml), washed with 0.1 N hydrochloric acid and with brine, and dried (MgSO₄). After concentration to dryness, the residue is taken up with ethyl ether (100 ml) and filtered, to give, after drying, a solid (21 g).

Purification by flash chromatography, elution being carried out with a 90-10 CH₂Cl₂-EtOAc mixture, gives, after elimination of the solvent and drying, compound 1 (13.4 g).

White crystals

Mp=115° C.

α_{D}⁵=12.90 (EtOH; c=0.46)

TLC: Merck silica gel 60 F254

Rf: 0.87 (70-30 CH₂Cl₂-EtOAc)

NMR (DMSO d₆) δ: 0.85 (t, 3H); 1.2-1.4 (m, 18H); 1.60 (m, 2H); 1.95 (s, 3H); 2.09 (s, 3H); 2.11 (s, 3H); 2.98-3.25 (m, 2H); 5.42 (s, 1H); 6.74 (s, 1H), 7.4-7.5 (m, 3H); 7.6-7.7 (m, 2H), 8.15 (s, 1H); 9.77 (s, 1H).

### Example 2

### (S)-2′,3′,5′-Trimethyl-4′-hydroxy-α-(12,12-difluoro-dodecylsulfonyl)-α-phenylacetanilide 2

### a) 12,12-Difluoro-1-bromododecane 2a

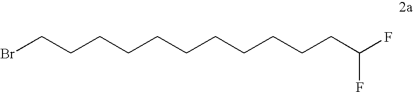

A solution of 12-bromo-1-decanol (12.31 g; 0.046 mol) in dichloromethane (70 ml) is added rapidly to a solution of pyridinium chlorochromate (14.2 g; 0.066 mol) in dichloromethane (90 ml). After stirring at ambient temperature for 5 hours, the reaction mixture is abundantly diluted with ethyl ether and filtered through celite. After evaporation and purification on silica, elution being carried out with a 5-95 EtOAc-petroleum ether mixture, crude 12-bromododecanal (8.74 g) is obtained.

The aldehyde (8.74 g; 0.033 mol) is taken up in methylene chloride (170 ml) and diethyl aminosulfide trifluoride (DAST) (5.3 ml; 0.04 mol) in methylene chloride (120 ml) is added dropwise thereto.

After reaction at ambient temperature for 4 hours, the mixture is concentrated to dryness and taken up with ethyl acetate, and washed with water and then with brine. After drying (MgSO₄), filtration and evaporation of the solvent, a dark oil is obtained, which is purified by chromatography on silica. By means of elution with petroleum ether, compound 2a (6.18 g) is obtained.

TLC: Merck silica gel 60 F254

Rf=0.27 (petroleum ether)

### b) (S)-α-(12,12-Difluorododecylthio)phenylacetic acid 2b

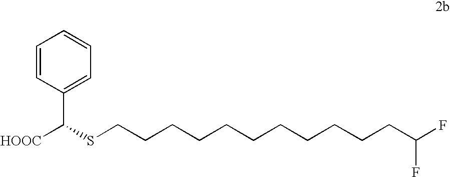

A solution of compound 2a (6.18 g; 0.022 mol) in ethanol (15 ml) is added to a solution of (S)-thiomandelic acid (3.04 g; 0.018 mol) in ethanol (70 ml), followed by sodium bicarbonate (3.64 g) in water (70 ml), in small portions.

After reaction for 7 hours at reflux, the ethanol is evaporated off. The solution is then acidified (1N HCl) and then extracted with ethyl acetate.

After drying (MgSO₄), filtration and evaporation to dryness, an oil is recovered, which is purified by flash chromatography. By means of elution with a 98-2 CH₂Cl₂-MeOH mixture, compound 2b (4.0 g) is obtained after elimination of the solvent.

Mp=48° C.

TLC=Merck silica gel 60 F254
Rf=0.34 (95-5 CH₂Cl₂-MeOH)

### c) (S)-2′,3′,5′-Trimethyl-4′-hydroxy-α-(12,12-difluorododecylthio)-α-phenylacetanilide

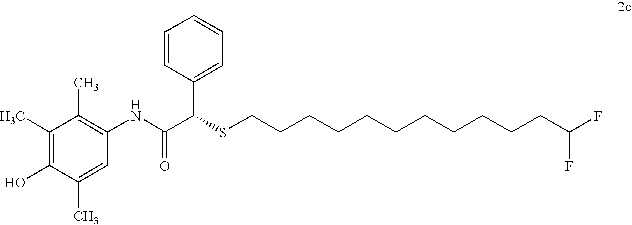

Triethylamine (1.33 ml) and then a solution of compound 2b (3.8 g; 0.01 mol) in dichloromethane (45 ml) and dicyclohexylcarbodiimide (2.2 g, 0.01 mol) are added to a solution of 2,3,5-trimethyl-4-aminophenol hydrochloride (1.76 g; 0.0095 mol) in dichloromethane (100 ml), maintained under nitrogen.

After 8 hours at ambient temperature with stirring, the dicyclohexylurea formed is filtered and the filtrate is concentrated to dryness and then taken up with ethyl acetate.

After washing with 0/1N hydrochloric acid and with water, drying (MgSO₄), and then evaporation under vacuum, a red solid is obtained, which is purified by flash chromatography.

Elution with an EtOAc-petroleum ether mixture gives, after evaporation of the solvent, compound 2c (4.12 g).

TLC: Merck silica gel 60 F254
Rf=0.2 (30-70 EtOAc-petroleum ether).

### d) (S)-2′,3′,5′-Trimethyl-4′-hydroxy-α-(12,12-difluorododecylsulfonyl)-α-phenylacetanilide

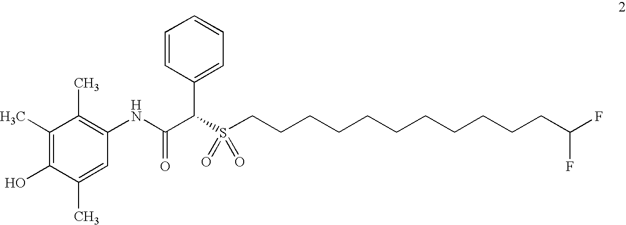

This compound is prepared according to the process described in example 1 using compound 2c obtained above.

White crystals

Mp=106° C.

α_{D}²⁵=+20° C. (EtOH; c=0.310)

TLC: Merck silica gel 60 F254
Rf=0.46 (30-70 EtOAc-petroleum ether)

NMR (DMSO d₆) δ: 1.20-1.35 (m, 18H); 1.6 (m, 2H); 1.95 (s, 3H); 2.09 (s, 3H); 2.11 (s, 3H); 2.98-3.25 (m, 2H); 5.42 (s, 1H); 6.03 (t, 1H); 6.74 (s, 1H); 7.4-7.5 (m, 3H); 7.6-7.7 (m, 2H); 8.15 (s, 1H); 9.78 (s, 1H).

### Example 3

### 2′,3′,5′-Trimethyl-4′-hydroxy-α-dodecylsulfonyl-α-fluoro-α-phenylacetanilide

### a) Methyl α-dodecylsulfonylphenylacetate 3a

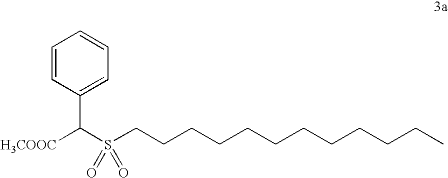

m-Chloroperbenzoic acid (11.53 g; 0.05 mol) is added slowly to a solution of methyl α-dodecylthiophenylacetate (8.6 g, 0.025 mol) in dichloromethane (120 ml).

After 2 hours at ambient temperature with stirring, the reaction mixture is filtered and evaporated. The residue obtained is purified by flash chromatography.

Elution with an EtOAc-petroleum ether mixture gives, after evaporation of the solvent, compound 3a (7.62 g).

Mp=59° C.

TLC: Merck silica gel 60 F254
Rf=0.45 (20-80 EtOAc-petroleum ether).

### b) Methyl α-fluoro-α-dodecylsulfonylphenylacetate 3b 3b

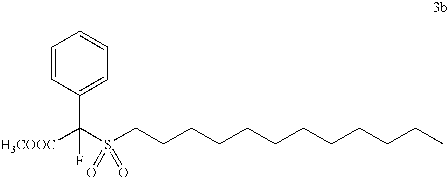

A solution of compound 3a (7.62 g; 0.02 mol) in THF (200 ml) is added, while maintaining the temperature below 7° C., to a suspension of sodium hydride (0.8 g; 0.02 mol) in THF (50 ml), at 0° C. under nitrogen.

After 30 minutes at 0° C. and 30 minutes at ambient temperature, DMF (20 ml) and select-fluor (7.07 g; 0.02 mol) are added, and then the mixture is maintained for 5 hours at ambient temperature with stirring.

The residue, obtained after evaporation of the THF, is taken up with N hydrochloric acid and extracted with ethyl acetate. After washing with water and with brine and drying (MgSO₄), an oil is obtained, after evaporation, which oil is purified by flash chromatography.

Elution with an EtOAc-petroleum ether mixture gives, after elimination of the solvent, compound 3b (6.49 g).

TLC: Merck silica gel 60 F254
Rf=0.37 (10-90 EtOAc-petroleum ether).

### c) α-Fluoro-α-dodecylsulfonylphenylacetic acid 3c

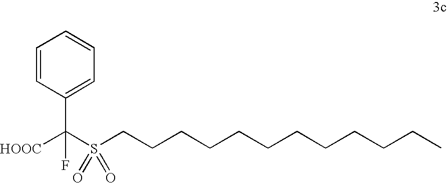

1N sodium hydroxide (31.7 ml) is added to a solution of compound 3b (6.49 g; 0.016 mol) in ethanol (160 ml).

After 2 hours at ambient temperature, with stirring, the methanol is evaporated off and the concentrate is acidified with 1N hydrochloric acid and then extracted with ethyl acetate.

After drying (MgSO₄) and evaporation of the solvent, an oil is recovered, which is taken up with petroleum ether. The crystals formed are filtered off and dried, to give compound 3c.

TLC: Merck silica gel 60 F254
Rf=0.3 (85-15 CH₂Cl₂ MeOH)

### d) 2′,3′,5′-Trimethyl-4′-hydroxy-α-dodecylsulfonyl-α-fluoro-α-phenylacetanilide 3

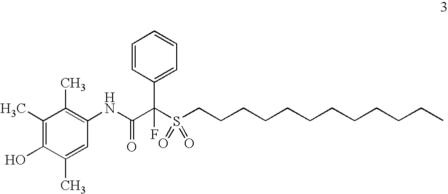

This compound is prepared according to the process described in example 2c using compound 3c obtained above instead of compound 2b.

Off-white crystals

Mp=81° C.

TLC: Merck silica gel 60 F254
Rf=0.23 (20-80 EtOAc-petroleum ether).

NMR (DMSO d₆) δ: 0.85 (t, 3H), 1.19-1.35 (m, 18H); 1.60 (m, 2H); 1.92 (s, 3H); 2.09 (s, 3H); 2.11 (s, 3H); 3.1-3.30 (m, 2H); 6.65 (s, 1H); 7.53-7.59 (m, 3H); 7.82-7.84 (m, 2H); 8.21 (s, 1H); 10.24 (S, 1H).

### Example 4

### 2′,3′,5′-Trimethyl-4′-hydroxy-α-(2-dodecyl-2H-5-tetrazolyl)-α-phenylacetanilide 4

### a) Ethyl α-(2H-5-tetrazolyl)phenylacetate 4a

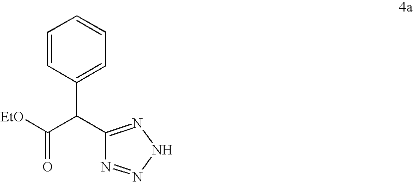

Trimethylsilyl azide (22.6 mg; 0.17 mol) and then dibutyl tin oxide (2.49 g; 0.01 mol) are added to a solution of ethyl phenylcyanoacetate (17.4 ml, 0.1 mol) in toluene (225 ml), and the reaction mixture is heated at 85° C. for 6 hours.

After evaporation of the toluene, the oily residue is taken up with ethanol (200 ml) and then once again evaporated. The residue is taken up with ethyl acetate. The solution is washed with 1N hydrochloric acid, with water, and then with brine, and the solution is dried (Na₂SO₄) and evaporated under vacuum, to give an oil which crystallizes from ethyl ether (16 g).

Mp=107-108° C.

TLC: Merck silica gel 60 F254
Rf=0.42 (90-10 CH₂Cl₂-MeOH)

### b) Ethyl α-(2-dodecyl-2H-5-tetrazolyl)phenylacetate 4b

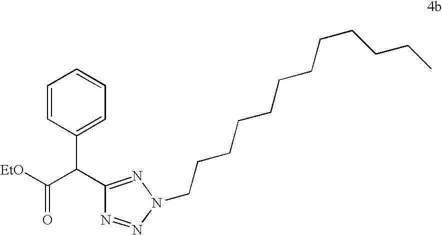

A solution of compound 4a (13.9 g; 0.06 mol), of triethylamine (16.7 ml; 0.12 mol) and of dodecyl bromide (15.8 ml; 0.066 mol) in acetonitrile (250 ml) is refluxed for 20 hours. After evaporation of the solvent under vacuum, the residue is taken up with ethyl acetate and the triethylene hydrobromide is eliminated by filtration. The filtrate is concentrated and purified by flash chromatography. By means of elution with a 10-90 EtOAc-petroleum ether mixture, the oily compound 4b (16.5 g) is obtained after elimination of the solvent.

TLC: Merck silica gel 60 F254
Rf=0.24 (5-95 EtOAc-petroleum ether).

### c) α-(2-Dodecyl-2H-5-tetrazolyl)phenylacetic acid 4c 4c

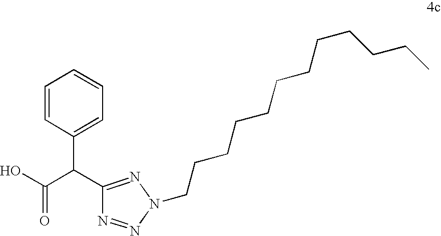

Sodium hydroxide pellets (2 g; 0.05 mol) are added to a solution of compound 4b (10 g; 0.025 mol) in ethanol (100 ml), and the mixture is stirred at ambient temperature for 5 hours. After concentration to dryness, the residue is taken up with water, acidified with 1N hydrochloric acid, and extracted with ethyl ether. The organic phase, washed with water, is dried (Na₂SO₄) and concentrated under vacuum, to give an oil that crystallizes from petroleum ether (8.9 g).

Mp=58° C.

TLC: Merck silica gel 60 F254
Rf=0.38 (95-5 CH₂Cl₂-MeOH)

### d) 2′,3′,5′-Trimethyl-4′-hydroxy-α-(2-dodecyl-2H-5-tetrazolyl)-α-phenylacetanilide 4

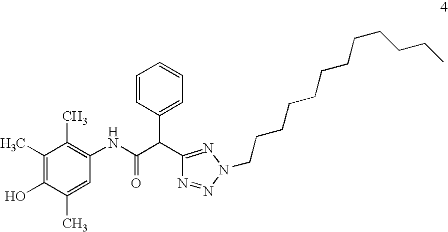

This compound is prepared according to the process described in example 2c using compound 4c obtained above instead of compound 2b.

White crystals

Mp=94° C.

TLC: Merck silica gel 60 F254
Rf=0.64 (50-50 EtOAc-hexane).

NMR (DMSO d₆) δ: 0.84 (t, 3H), 1.21-1.34 (m, 18H); 1.87 (m, 5H); 2.06 (s, 3H); 2.08 (s, 3H); 4.58 (t, 2H); 5.5 (s, 1H); 6.7 (s, 1H); 7.25-7.40 (m, 3H); 7.51-7.53 (m, 2H); 8.06 (s, 1H); 9.60 (s, 1H).

### Example 5

### (+)-2′,3′,5′-Trimethyl-4′-hydroxy-α-(2-dodecyl-2H-5-tetrazolyl)-α-phenylacetanilide 5

Compound 4 (23.9 g) is taken up in a minimum amount of ethanol and chromatographed on a chiral pack AD column. By means of elution with a 20-80 EtOH-hexane mixture, compound 5 (10.9 g) is obtained after evaporation of the solvent.

White crystals

Mp=105° C.

α_{D}²⁵=42.30 (EtOH; c=0.362).

### Example 6

### (+)-2′,3′,5′-Trimethyl-4′-hydroxy-α-(2-hexyl-2H-5-tetrazolyl)-α-phenylacetanilide 6

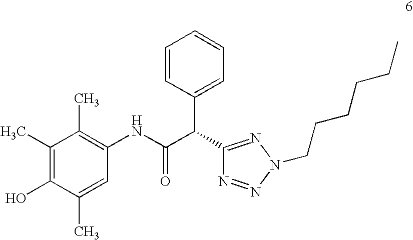

This compound is obtained according to the process described in example 4, by replacing, in stage 4b, the dodecyl bromide with hexyl bromide, and is then resolved according to the process described in example 5, elution being carried out with a 70-30 hexane-ethanol mixture.

White crystals

Mp=108° C.

Merck silica gel 60 F254
Rf=0.14 (10-90 EtOAc-petroleum ether).

NMR (DMSO d₆) δ: 0.84 (t, 3H); 1.24 (m, 6H); 1.87 (m, 5H); 7.06 (s, 3H); 2.08 (s, 3H); 4.64 (t, 2H); 5.5 (s, 1H) 6.7 (s, 1H); 7.29-7.39 (m, 3H); 7.51-7.53 (m, 2H), 8.05 (s, 1H); 9.60 (s, 1H).

### Example 7

### 2′,3′,5′-Trimethyl-4′-hydroxy-α-(2-decyl-2H-5-tetrazolyl)-α-phenylacetanilide 7

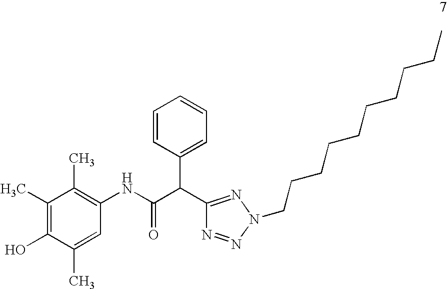

This compound is obtained according to the process described in example 4, by replacing, at stage 4b, the dodecyl bromide with decyl bromide.

White crystals

Mp=87° C.

TLC: Merck silica gel 60 F254
Rf=0.71 (80-20 CH₂Cl₂-EtOAc)

### Example 8

### 2′,3′,5′-Trimethyl-4′-hydroxy-α-[(2-(6,6-difluoro-hexyl)-2H-tetrazolyl)-α-phenylacetanilide 8

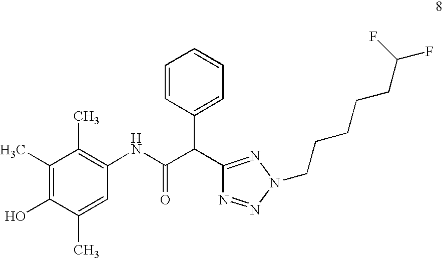

This compound is obtained according to the process described in example 4, by replacing, at stage 4b, the dodecyl bromide with 1-bromo-6,6-difluorohexane, itself obtained according to example 2a by replacing the 12-bromodecanol with 6-bromohexanol.

White crystals

Mp=120° C.

Merck silica gel 60 F254
Rf=0.53 (70-30 CH₂Cl₂-EtOAc)

NMR (DMSO d₆) δ: 1.26-1.41 (m, 4H); 1.75-1.90 (m, 4H); 1.92 (s, 3H); 2.06 (s, 3H); 2.08 (s, 3H); 4.65 (t, 7H); 5.52 (s, 1H); 6.01 (t, 1H); 6.71 (s, 1H), 7.30-7.40 (m, 3H); 7.51-7.54 (m, 2H); 8.05 (s, 1H), 9.60 (s, 1H)

### Example 9

### (+)-21,3′,5′-Trimethyl-4′-hydroxy-α-(2-dodecyl-2H-5-tetrazolyl)-α-fluoro-α-phenylacetanilide 9

### a) Ethyl α-(2-dodecyl-2H-5-tetrazolyl)-α-fluorophenylacetate 9a

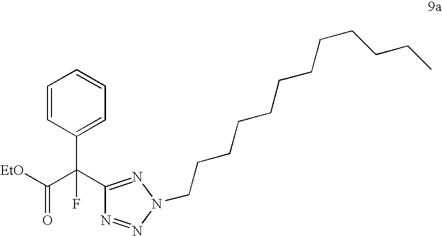

Compound 4b (10.65 g; 0.027 mol) in solution in THF (120 ml) is added dropwise to a suspension of sodium hydride (1.06 g; 0.027 mol) in THF (60 ml) at −8° C. under nitrogen. After 30 minutes, DMF (25 ml) and select-fluor (9.61 g; 0.027 mol) are added, and the stirring is maintained at ambient temperature for 20 hours.

The residue obtained after concentration under vacuum is taken up with ethyl ether, and washed with hydrochloric acid, with water and with brine. After drying (Na₂SO₄), the crude oily compound 9a (10.9 g) is obtained.

TLC: Merck silica gel 60 F254
Rf=0.66 (5-95 EtOAc-petroleum ether).

### b) α-(2-dodecyl-2H-5-tetrazolyl)-α-fluorophenylacetic acid 9b

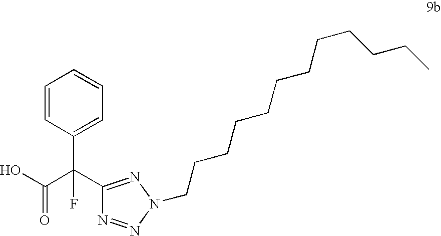

This compound is obtained according to the process described in example 4c, starting from compound 9b obtained above.

TLC=Merck silica gel 60 F254.
Rf=0.45 (85-15 CH₂Cl₂-MeOH)

### c) (+)-α-(2-Dodecyl-2H-5-tetrazolyl)-α-fluorophenyl-acetic acid 9c

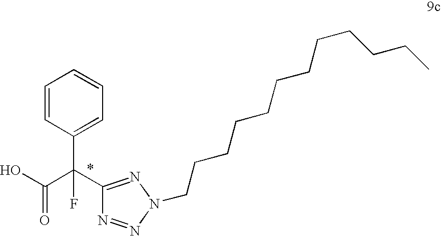

Isobutyl chloroformate (13.3 ml; 0.1 mol) and then N-methylmorpholine (11.5 ml; 0.1 mol) are added to a solution of compound 9b (35 g; 0.09 mol) in dichloromethane (300 ml), maintained at −10° C. After stirring for 30 minutes, (+)-norephedrine is added and the mixture is stirred at ambient temperature for 3 hours. The reaction mixture is washed with water, with aqueous sodium bicarbonate and with brine, and then dried (Na₂SO₄) and concentrated under vacuum.

The diastereoisomeric amides thus obtained are separated by flash chromatography. By means of elution with a 20-80 EtOAc-petroleum ether mixture, the least polar amide is isolated (14.9 g) and is treated with concentrated hydrochloric acid (300 ml) in dioxane (300 ml). After stirring at reflux for 3 hours, the mixture is concentrated and then taken up with dichloromethane, and then washed with water, with 1N hydrochloric acid and with brine. After drying (Na₂SO₄) and elimination of the solvent under vacuum, compound 9c is obtained.

### d) (+)-2′,3′,5′-Trimethyl-4′-hydroxy-α-(2-dodecyl-2H-tetrazolyl)-α-fluoro-α-phenylacetanilide 9

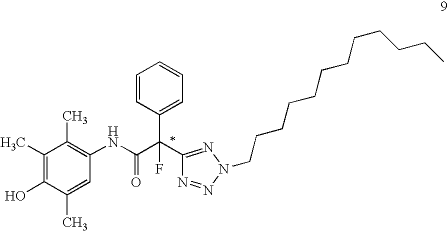

This compound is prepared according to the process described in example 2c using compound 9c obtained above instead of compound 2b.

White crystals

Mp=126° C.

α_{D}²⁵=66.10 (EtOH; c=0.31)

TLC: Merck silica gel 60 F254
Rf=0.40 (EtOAc).

NMR (DMSO d₆) δ: 0.85 (t, is); 1.23 (m, 18H); 1.90 (m, 2H); 1.92 (s, 3H); 2.08 (s, 3H); 2.11 (s, 3H); 4.71 (t, 2H); 6.67 (s, 1H); 7.48-7.51 (m, 3H); 7.59-7.62 (m, 2H), 8.13 (s, 1H); 10.17 (s, 1H).

### Example 10

### 2′,3′,5′-Trimethyl-4′-hydroxy-α-[2-(12,12-difluoro-dodecyl)-2H-5-tetrazolyl]-α-fluoro-α-phenylacetanilide 10

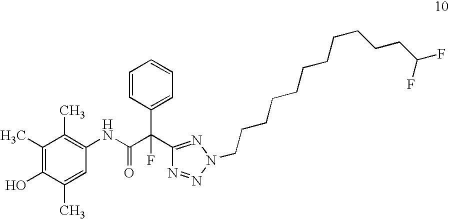

This compound is prepared according to the process described in example 4b, by replacing the dodecyl bromide with 1-bromo-12,12-difluorododecane obtained as described in example 2a. The intermediate compound thus obtained is treated according to the process described in example 9a,b,d, to give compound 10.

White crystals

Mp=96° C.

TLC: Merck silica gel 60 F254
Rf=0.44 (30-70 EtOAc-petroleum ether).

NMR (DMSO d₆) δ: 1.22-1.35 (m, 16H); 1.76-1.78 (m, 2H); 1.79-1.92 (m; 5H); 2.08 (s, 3H); 2.11 (s, 3H); 4.72 (t, 2H); 6.03 (t, 1H); 6.67 (s, 1H); 7.48-7.50 (m, 3H); 7.60-7.62 (m, 2H); 8.13 (s, 1H); 10.06 (s, 1H).

### Example 11

### 2′,3′,5′,6′-Tetramethyl-4′-amino-α-(2-dodecyl-2H-5-tetrazolyl)-α-fluoro-α-phenylacetanilide; hydrochloride

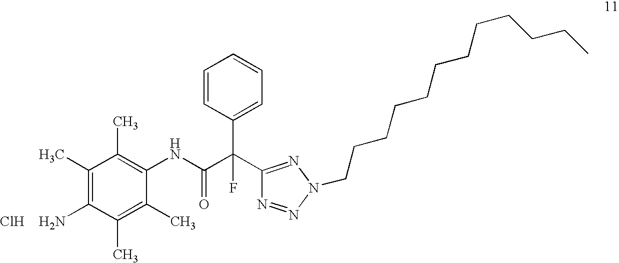

Compound 9b (0.80 g; 0.002 mol), obtained in example 9, in solution in THF (5 ml) at 0° C. under nitrogen is treated dropwise with a solution of oxalyl chloride (0.2 ml) in THF (5 ml). After 4 hours at ambient temperature with stirring, the reaction mixture is added dropwise to a solution of diisopropylethylamine (0.42 ml) and of 2,3,5,6-tetramethyl-1,4-phenylenediamine (0.37 g; 0.0022 mol) in THF, maintained under nitrogen.

After stirring for 3 hours, the mixture is concentrated under vacuum, taken up with ethyl acetate, and washed with water and with brine. After drying (MgSO₄) and elimination of the solvent under vacuum, an oil is recovered, which is purified by flash chromatography, elution being carried out with a 95-5 CH₂Cl₂-EtOAc mixture.

The eluant is concentrated under vacuum, taken up with acetone (10 ml) and treated with 3.16 N hydrochloric acid in isopropanol (0.18 ml).

The precipitate formed is filtered off, washed with ethyl ether and dried, to give compound 11 (220 mg).

White crystals

Mp=168° C.

TLC: Merck silica gel 60 F254
Rf=0.20 (95-5 CH₂Cl₂-EtOAc-petroleum ether).

NMR (DMSO d₆) δ: 0.85 (t, 3H); 1.23 (m, 18H); 1.94 (s, 3H); 1.88-1.92 (m, 2H); 1.99 (s, 3H); 2.05 (s, 3H); 2.07 (s, 3H); 4.73 (t, 2H); 7.49-7.50 (m, 3H); 7.61-7.63 (m, 2H); 10.28 (s, 1H).

### Example 12

### 2′,3′,5′,6′-Tetramethyl-4′-amino-α-(2-hexyl-2H-5-tetrazolyl)-α-phenylacetanilide hydrochloride 12

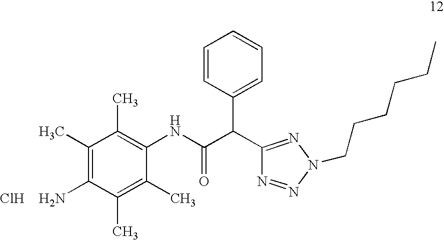

This compound is obtained according to the process described in example 2c, by replacing the 2,3,5-trimethylaminophenol with 2,3,5,6-tetramethylphenylenediamine, and the x-(12,12-difluorododecylthio)phenylacetic acid with α-(2-hexyl-2H-5-tetrazolyl)phenylacetic acid.

After salification with hydrochloric acid, in isopropanol, compound 12 is obtained by precipitation with ethyl ether.

White crystals

Mp=252° C.

TLC: Merck silica gel 60 F254
Rf=0.48 (80-20 CH₂Cl₂-EtOAc)

The compounds of the invention were subjected to pharmacological trials which showed their potential advantage in the treatment of hypercholesterolemia and in the treatment of atheromatous disease.

The compounds were studied for their ACAT-inhibiting effect in vitro and blood cholesterol-lowering effect in rats.

1—ACAT Inhibition

the ACAT (acyl COA: cholesterol O-acyl transferase enzyme) inhibiting activity of the compounds was evaluated in vitro on rat liver microsomes using the technique of H. Chautan et al. (Analytical Biochemistry, 173, 436-439, 1988).

The activities, expressed as 50% inhibitory concentrations (IC 50) obtained with certain products of the invention and eflucimibe (example 16 of patent WO 97/19918 filed by the applicant) are reported by way of example in table I below:

| Compound No. | IC₅₀ (nμ) |
|---|---|
| 1 | 135 |
| 3 | 48 |
| 4 | 43 |
| 5 | 11 |
| 9 | 20 |
| 10 | 28 |
| Eflucimibe | 60 |

2—Blood Cholesterol-Lowering Activity

Male rats (160-180 g) were subjected, for 4 days, to an Altromin C 1061 hypercholesterolemic diet and treated in parallel orally with the compounds in suspension in a solution of 2% Tween 80 in distilled water.

On the 5^{th} day, the animals not fasting are anaesthetized with ethyl ether, and bled out on EDTA via the abdominal aorta. The blood is immediately centrifuged and the plasma is stored at 4° C.

The plasma cholesterol is then assayed by the CHOD-PAP method (Boehringer Mannheim Ref. 237574). The 50% effective dose (ED₅₀) corresponds to the dose that reduces the plasma cholesterol concentration by half compared with control animals.

| | |
|---|---|
| Compound No. | ED₅₀ (mg/kg) |

| | |
|---|---|
| 1 | 0.25 |
| 3 | 0.022 |
| 4 | 0.029 |
| 5 | 0.025 |
| 9 | 0.012 |
| 10 | 0.029 |
| Eflucimibe | 0.12 |

the compounds of the invention are powerful ACAT-inhibiting blood cholesterol-lowering agents which can be used in the treatment of diseases such as hypercholesterolemia and atherosclerosis.

The pharmaceutical compositions can be provided in the form suitable for oral, parenteral or local administration, for example in the form of capsules, tablets, granules, gelatin capsules, liquid solids, syrups or oral suspensions, and may contain the appropriate excipients.

The daily dosage can range from 5 to 1000 mg.

1. An anilide derivative having the general formula I: 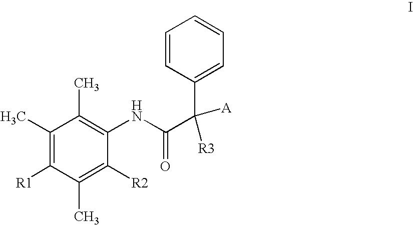
wherein:
R₁ is a hydroxyl or amino group;
R₂ is a hydrogen or methyl radical;
R₃ is a hydrogen or a fluorine atom; and,
wherein A is 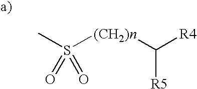
or 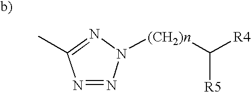
wherein:
n is an integer from 5 to 11, limits inclusive, and
R₄ and R₅, are independently a hydrogen or a fluorine atom; and
enantiomers or stereoisomers of said analide derivative, and pharmaceutically acceptable salts thereof.

2. An analide derivative in accordance with claim 1—selected from the group consisting of:
(S)-2′,3′,5′-trimethyl-4′-hydroxy-α-dodecylsulfonyl-α-phenylacetanilide;
(S)-2′,3′,5′-trimethyl-4′-hydroxy-α-(12,12-difluorododecylsulfonyl)-α-phenylacetanilide;
2′,3′,5′-trimethyl-4′-hydroxy-α-dodecylsulfonyl-α-fluoro-α-phenylacetanilide;
2′,3′,5′-trimethyl-4′-hydroxy-α-(2-dodecyl-2H-5-tetrazolyl)-α-phenylacetanilide;
(+)-2′,3′,5′-trimethyl-4′-hydroxy-α-(2-dodecyl-2H-5-tetrazolyl)-α-phenylacetanilide:
(+)-2′,3′,5′-trimethyl-4′-hydroxy-α-(2-hexyl-2H-5-tetrazolyl)-α-phenylacetanilide.
2′,3′,5′-trimethyl-4′-hydroxy-α-(2-decyl-2H-tetrazolyl)-α-phenylacetanilide,
2′,3′,5′-trimethyl-4′-hydroxy-α-[(2-(6,6-difluorohexyl)-2H-tetrazolyl]-α-phenylacetanilide;
(+)-2′,3′,5′-trimethyl-4′-hydroxy-α-(2-dodecyl-2H-5-tetrazolyl)-α-fluoro-α-phenylacetanilide;
2′,3′,5′-trimethyl-4′-hydroxy-α-[2-(12,12-difluorododecyl)-2H-5-tetrazolyl]-α-fluoro-α-phenylacetanilide;
2′,3′,5′,6′-tetramethyl-4′-amino-α-(2-dodecyl-2H-5-tetrazolyl)-α-fluoro-α-phenylacetanilide hydrochloride; and
2′,3′,5′,6′-tetramethyl-4′-amino-α-(2-hexyl-2H-5-tetrazolyl)-α-phenylacetanilide hydrochloride; and
enantiomers or stereoisomers of said analide derivatives, and pharmaceutically acceptable salts thereof.

3. A method for the treatment of hypercholesterolemia or atherosclerosis comprising administering to a patient the analide derivative of claim 1.

4. A pharmaceutical composition comprising an analide derivative of claim 1 and a pharmaceutically acceptable carrier.

5. A method for producing a medicinal products comprising the step of combining the analide derivative of claim 1 and a pharmaceutically acceptable carrier.

6. A method for the treatment of hypercholesterolemia or atherosclerosis comprising administering to a patient an analide derivative of claim 2.

7. A pharmaceutical composition comprising the analide derivative of claim 2 and a pharmaceutically acceptable carrier.

8. A composition comprising a mixture of two or more compounds of claim 1.

9. A method of lowering blood cholesterol comprising administering to a patient the analide derivative of claim 1.

10. A method of lowering blood cholesterol comprising administering to a patient the composition of claim 8.

11. A method for producing a medicinal product comprising the step of combining the analide derivative of claim 2 and a pharmaceutically acceptable carrier.
